(19) United States
(12) Plant Patent Application Publication
(10) Pub. No.: US 2020/0068765 P1
(43) Pub. Date: Feb. 27, 2020

(54) Pongamia tree named 'K207'

(50) ;

(71) Applicant: TerViva BioEnergy, Inc., Oakland, CA (US)

(72) Inventors: Naidu Bodapati, Riverhills (AU); Marshall MacKay, Tewantin (AU); Naveen Sikka, Oakland, CA (US); David Harry, Philomath, OR (US)

(73) Assignee: TerViva BioEnergy, Inc., Oakland, CA (US)

(21) Appl. No.: 16/602,152

(22) Filed: Aug. 14, 2019

Related U.S. Application Data
(60) Provisional application No. 62/722,102, filed on Aug. 23, 2018.

(51) Int. Cl. A01H 6/54

(52) U.S. Cl. PLT/216

(57) ABSTRACT

A new and distinct variety of Pongamia tree (Pongamia pinnata (L) Pierre) named ‘K207’ is provided herein. Pongamia tree ‘K207’2 is distinguished by having a dense canopy, large leaves with dark yellowish green color, semi-pendulous branches, unusually large lenticels on the stems, compact inflorescences, abundant production of seed pods, coupled with the high oil content of the seeds.

### CROSS-REFERENCE TO RELATED APPLICATION

This application claims the benefit of U.S. Provisional Application No. 62/722,102, filed Aug. 23, 2018, which is incorporated herein by reference in its entirety.

Latin name: Botanical classification: Pongamia pinnata (L) Pierre.

Varietal denomination: The varietal denomination of the claimed variety of Pongamia tree is ‘K207’.

### BACKGROUND OF THE INVENTION

Pongamia is a species of tree that belongs to the Fabaceae family of plants. The botanical classification of Pongamia is Pongamia pinnata (L) Pierre, although these plants are also known by a number of synonymous botanical classifications including, for example, Millettia pinnata (L) Panigrahi, Derris indica (Lam.) Bennet, Pongamia glabra Vent, and Cytisus pinnatus (L).

As a member of the Fabaceae family of plants, Pongamia trees are legumes and are capable of fixing their own nitrogen. Pongamia can grow well in a variety of environmental conditions, including areas with malnourished soil. Pongamia seeds also contain a profile of chemicals having a number of uses including, for example, commercial preparation into skin ointment and as a fuel source.

Pongamia trees are capable of growing in otherwise challenging environmental conditions and produce seed oils with beneficial properties. There exists a need for Pongamia varieties that are stable, high yielding, and agronomically sound.

### SUMMARY OF THE INVENTION

In order to meet these needs, the present disclosure is directed to an improved variety of Pongamia tree. In particular, the disclosure relates to a new and distinct variety of Pongamia tree (Pongamia pinnata (L) Pierre), which has been denominated as ‘K207’.

Pongamia tree ‘K207’ was discovered growing in a cultivated area in southeastern Queensland, Australia, wherein it and neighboring trees are grown for shade and other landscape amenities. The parentage of this tree is unknown, as is typical for trees cultivated in landscape plantings of this type.

The original donor tree (and source of clonal material) of ‘K207’ was identified and selected primarily on the basis of its consistent production of large and abundant seed pods, coupled with the high oil content (40.6%, dry-weight basis) of its large seeds. This selected individual was assigned identity number ‘K207’. Subsequently, branch cuttings were collected from the donor tree and rooted by treating the cuttings with the rooting hormone indolebutyric acid (IBA) using either an aqueous solution of IBA (500 ppm) or a commercially available rooting powder. Dehydration of the cuttings was avoided by placing them in moistened rooting medium within a humid enclosed chamber. Rooting success varies from one collection event to another, which is normal for cuttings from mature trees. Typically, 10% or more of the cuttings initiated root development within 3-4 weeks.

Pongamia tree ‘K207’ has been asexually reproduced via rooted cuttings in the Brisbane area of Queensland, Australia, as well as in Texas and Florida, U.S.A. In addition, shoots of ‘K207’ have also been grafted onto seedling rootstock using either a top-cleft graft or a side-veneer graft. These asexually propagated plants remain true-to-type, and show considerable resemblance among one another after accounting for variation in overall size of the propagated cuttings and seedling rootstocks.

Pongamia tree ‘K207’ is particularly and distinctly characterized by its very dense canopy, long current seasons semi-pendulous branchlets, unusually large oval to circular lenticels on stem, compact inflorescences, and bearing copious quantities of fruit pods containing seeds with high seed-oil content (40.6%, dry-weight basis). Pods tend to appear in dense clusters. ‘K207’ demonstrates excellent vigor and is reasonably resistant to insect and disease pests.

### DESCRIPTION OF THE DRAWINGS

Pongamia tree ‘K207’ is illustrated by the accompanying photographs, which show e.g. the plant's form, branches, foliage, leaves, trunk, pods, and seeds as specifically described below. The colors shown are as true as can be reasonably obtained by conventional photographic procedures.

FIG. 1 illustrates a view of the whole tree of Pongamia tree ‘K207’ in Queensland, Australia.

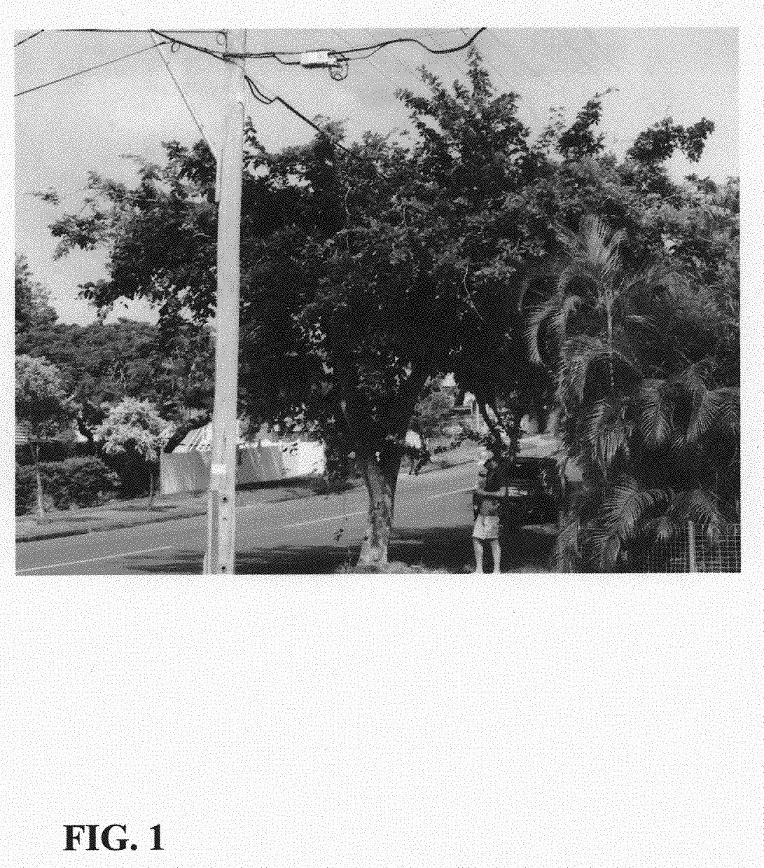

FIG. 2 illustrates a view of the leaves and seed pods on Pongamia tree ‘K207’ in Queensland, Australia.

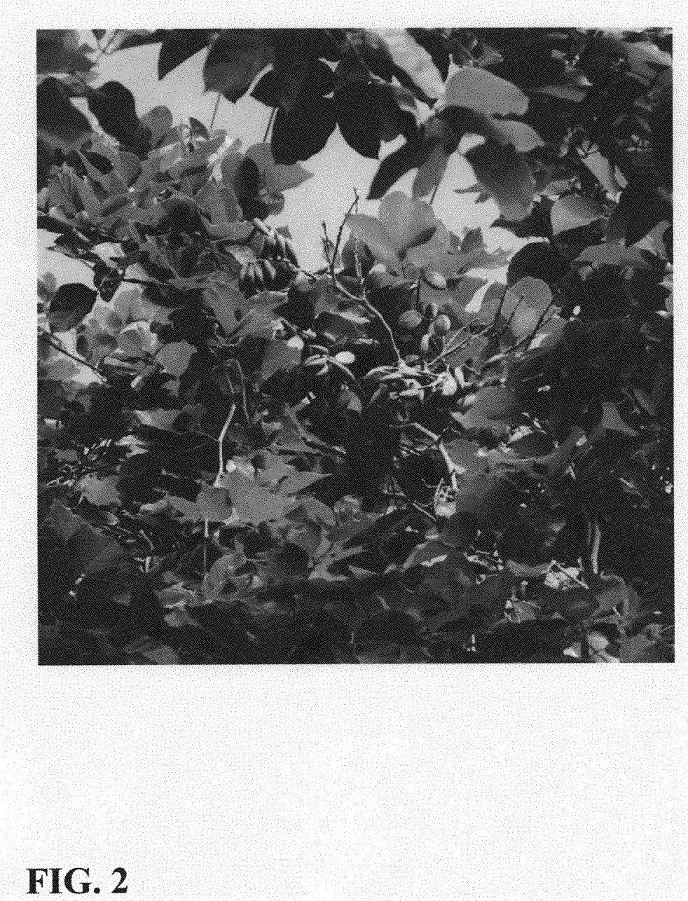

FIG. 3 illustrates a view of the leaves and seed pods on Pongamia tree ‘K207’ in Queensland, Australia.

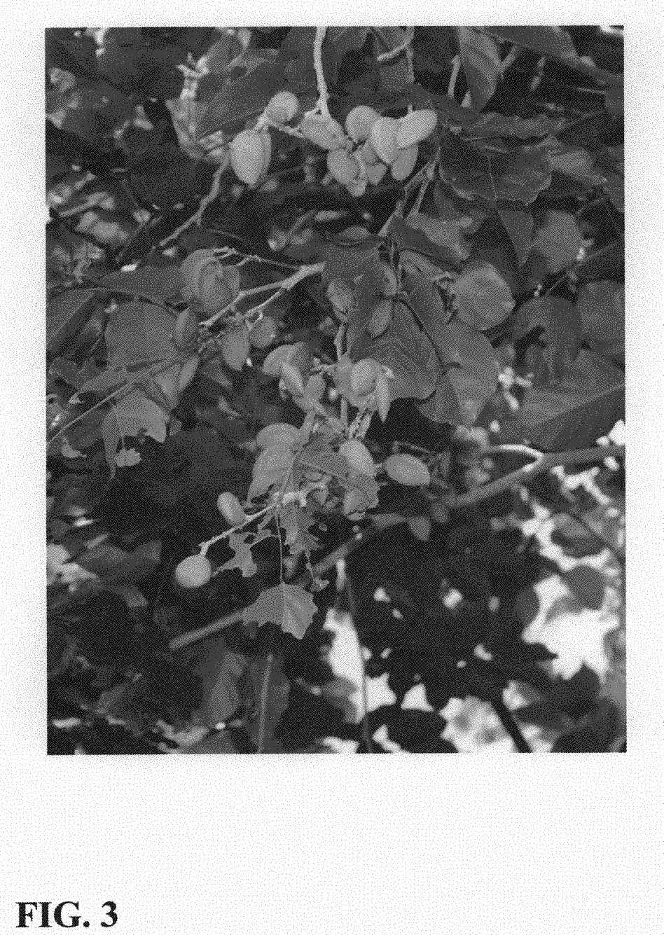

FIG. 4 illustrates a view of the tree trunk of Pongamia tree ‘K207’ in Queensland, Australia.

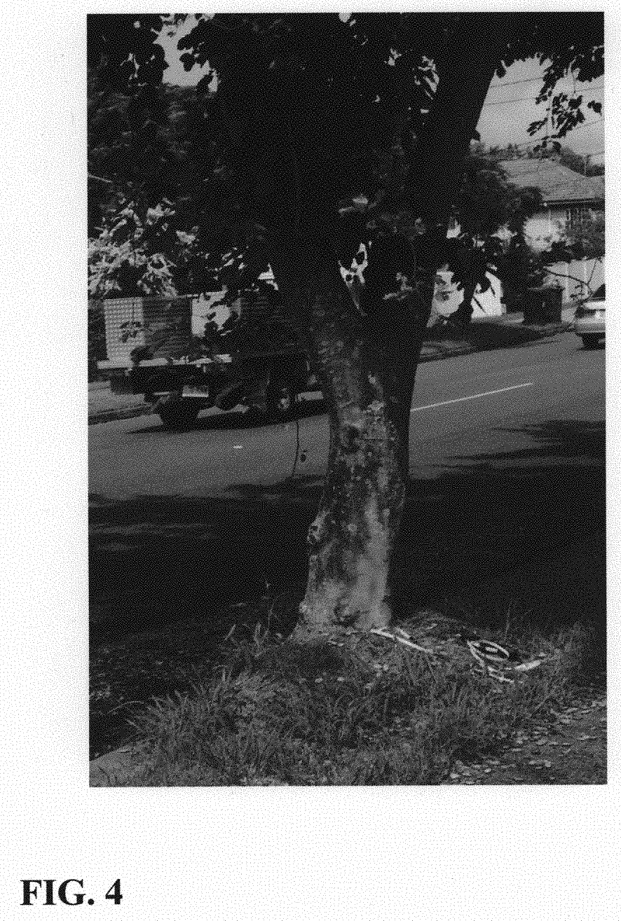

FIG. 5 illustrates a view of a 5-year-old clonally propagated ‘K207’ tree in Florida, USA.

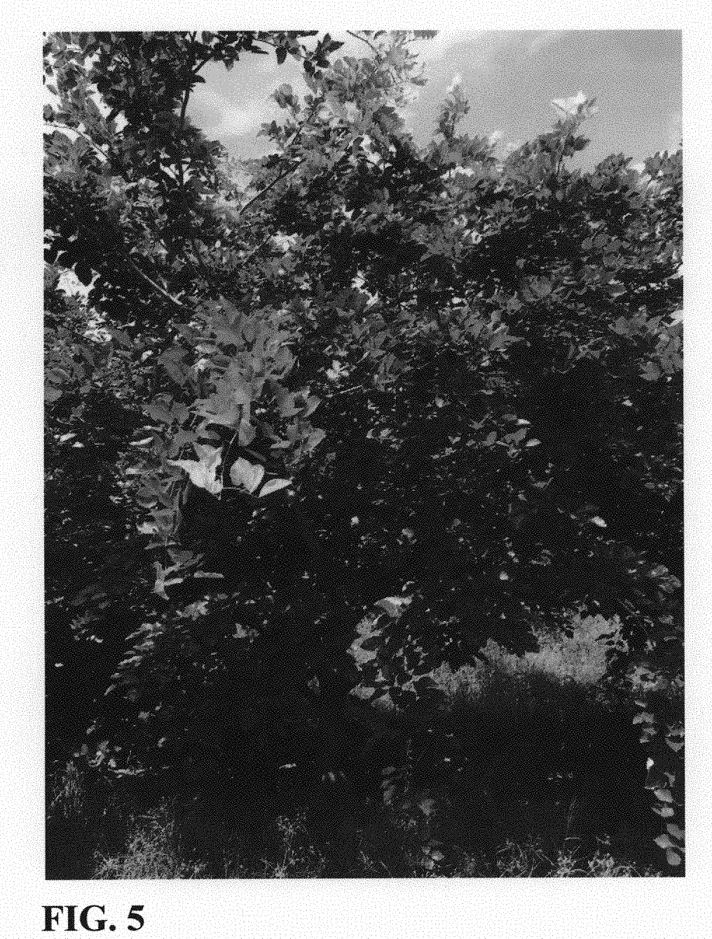

FIG. 6 illustrates a Florida-grown ‘K207’ tree with the smooth colored bark on the main trunk. Mottled patches on the bark primarily reflect differences in lichen growth. Unusually large stretched lenticular scars appear as horizontal striations along the bark.

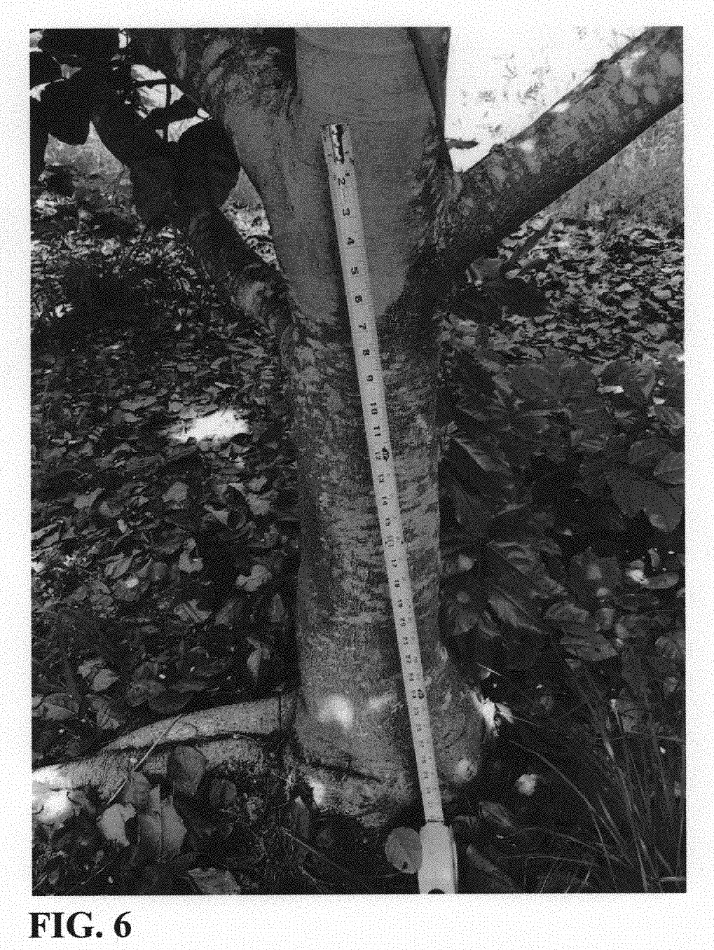

FIG. 7 illustrates the long current season's branch with odd-pinnately compound leaves from a Florida-grown ‘K207’ tree. The compound leaves typically bear a mix of leaves with either 5 (more common) or 7 leaflets. The leaf on the right is oriented to show the underside.

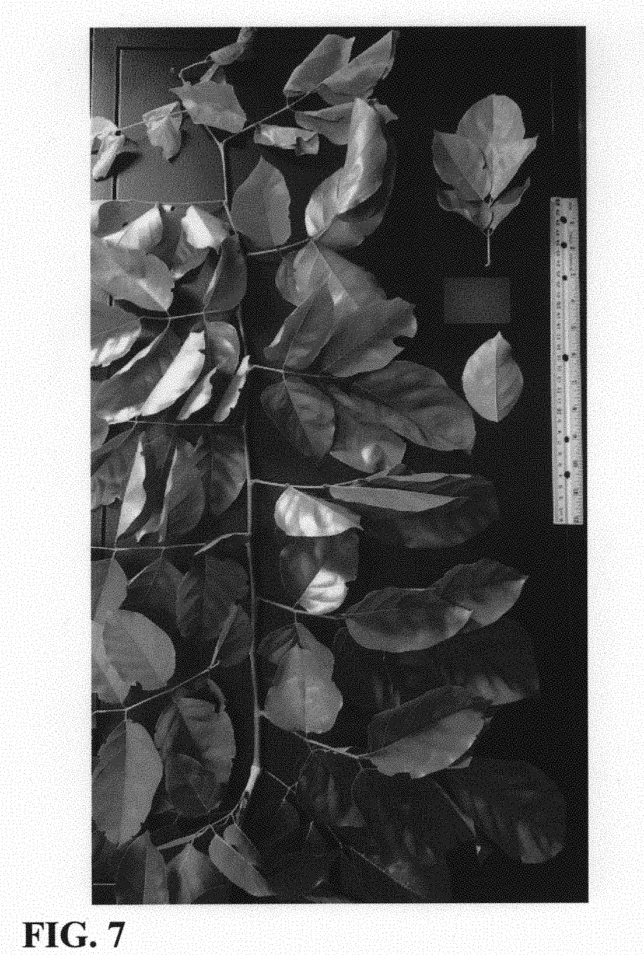

FIG. 8 illustrates the regular branching pattern arising from the leaf axils, and long current season's twigs on a Florida-grown ‘K207’ tree. Leaves have been removed for clarity.

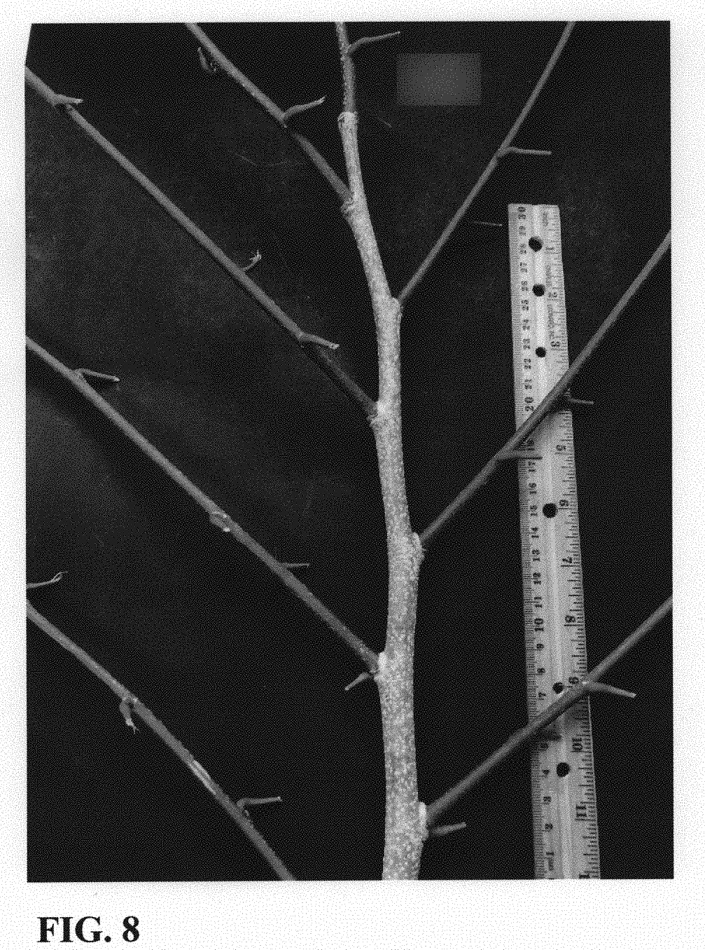

FIG. 9 illustrates the unusually large lenticels, especially on the previous season's wood, from a Florida-grown ‘K207’ tree.

FIG. 10 illustrates the compact inflorescences from a Florida-grown ‘K207’ tree.

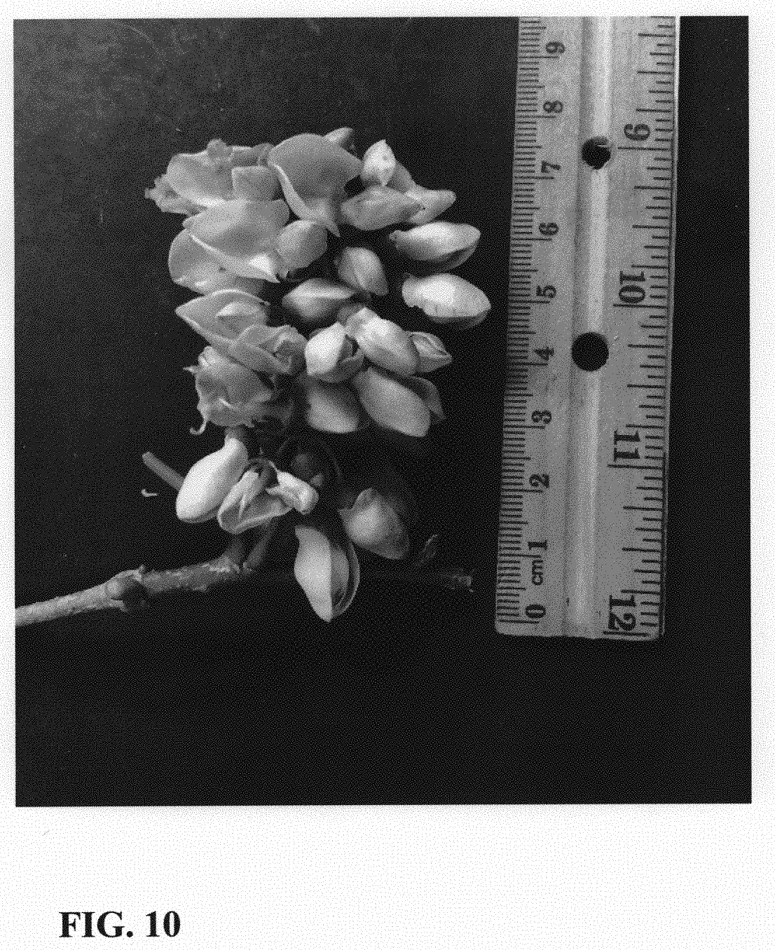

FIG. 11 illustrates semi-mature pods along with fully mature dry pods of Pongamia tree ‘K207’. Both pod stages have been split open to show seed. The branch shown has 2 rachises borne in the axils of basal leaves. Each rachis bears 3-6 immature pods.

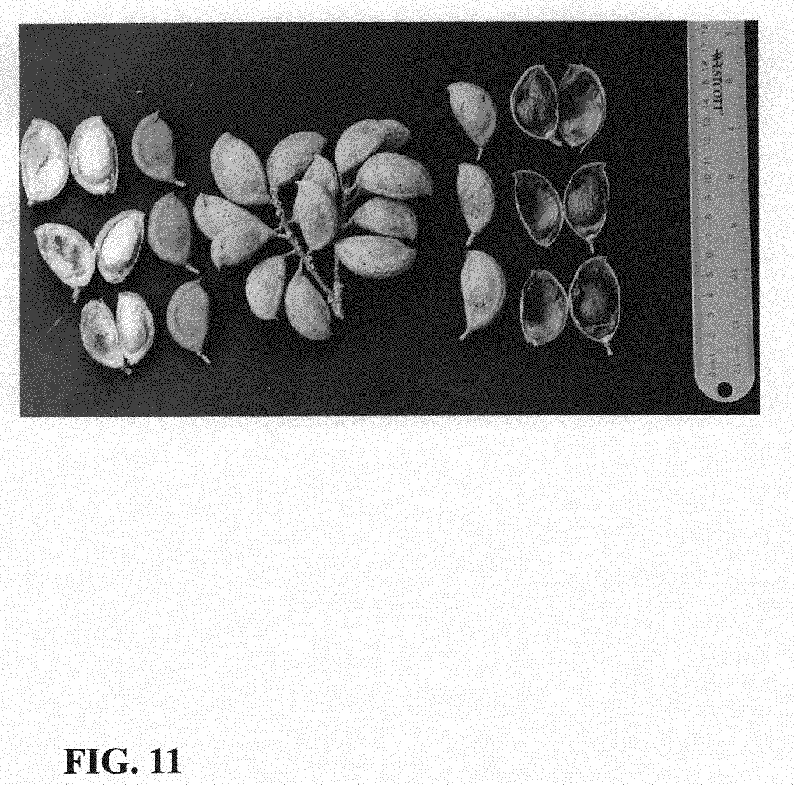

### DETAILED BOTANICAL DESCRIPTION OF THE PLANT

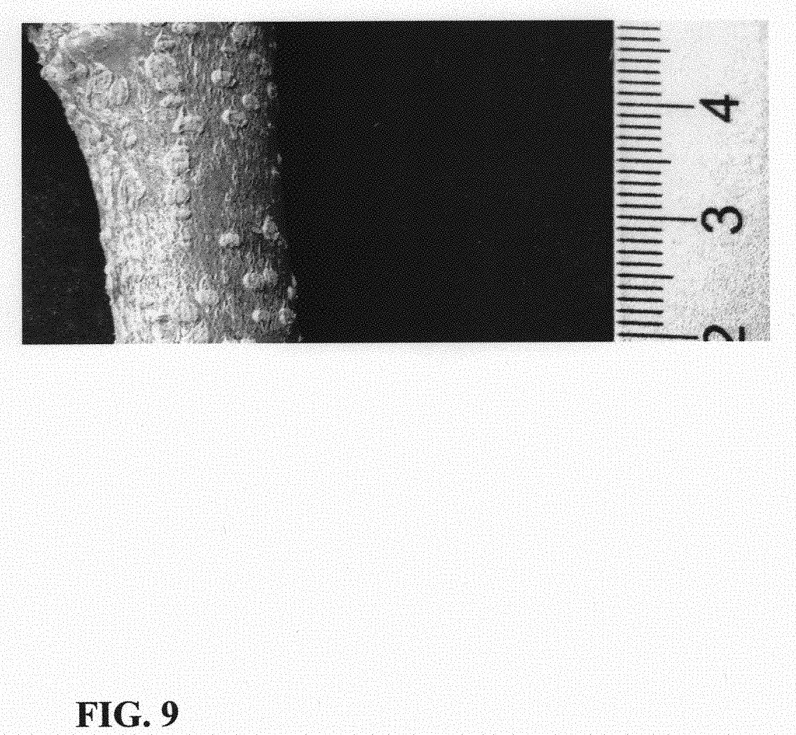

The following detailed description sets forth the phenotypic characteristics and the distinctive features of Pongamia tree ‘K207’. Descriptions are from trees that are 5 years post-transplant into the field in Florida, USA unless otherwise noted. Standardized color designations herein are with reference to The Royal Horticultural Society (R.H.S.) Mini Colour Chart.
Classification:
   Family.—Fabaceae.
   Botanical.—Pongamia pinnata (L) Pierre.
   Common name.—Pongam tree, pongam oil tree, or Pongamia.
Tree:
   Size.—Very vigorous tree of substantial size and stature, 8 m in height. Crown diameter, measured at the drip-line in two dimensions, is 6 m×6.5 m.
   Trunk.—The trunk is bifurcated at a height of 55 cm from the ground. The trunk can have numerous epicormic branches at its base (this trait is somewhat variable among different trees). The fresh vegetative growth on an epicormic shoot ranges from 25 cm to 60 cm long and from about 4.0 mm to 7.5 mm in diameter.
   Bark.—Greyish yellow green in color (RHS 196A) with smooth or faintly vertically fissured bark on the main trunk. Light-colored light greenish grey (RHS 190D) and dark-colored very pale green (RHS 192B) mottled patches on the bark reflect differences in lichen growth. Branchlets are hairless with pale stipule scars.
   Form.—Decurrent crown form with dense, compact foliage and the outer branchlets show semi-pendulous character.
Branches:
   Branch length.—Average breast-height diameter of the two major stems is 11 cm. The color of these branches closely resembles the color of the main stem.
   Branching habit.—Many branched limbs. Growth and size of lateral branches are considerably less than that of the main branches. Many smaller branches occur higher in the crown.
   Buds and shoots.—Young shoots emerge from naked lateral buds, with the most distal bud giving rise to a terminal shoot. Buds are hemispherical and range from 3.2 mm to 5.0 mm wide at their base. Buds protrude outward 2.0 mm to 3.0 mm from the stem and are between 1.5 mm to 2.0 mm thick. The typical axillary bud is strong yellow green in color (RHS 144A), and the younger terminal bud is also strong yellow green in color (RHS 144A).
   Terminal shoots.—The seasonal elongation of terminal shoots is considerable. Total elongation growth of the terminal shoots is an average of 45 cm and encompassing an average of 7 internodes. The average stem diameter of terminal shoots is 20.0 mm.
   Lateral shoots.—Seasonal elongation of lateral shoots is considerably long, with an average length of 55 cm and bearing 8 leaf nodes. The average diameter of lateral shoots is 5.3 mm.
   Shoot and twig surfaces.—Woody twigs have leaf scars that are 5.0 mm to 6.0 mm wide, showing traces of three vascular bundles. The central scar tends to have a border that is less distinct than the outer two. The youngest shoots are distinctly strong yellow green in color (RHS 143A) and flexible, with no lenticels apparent to the naked eye. As shoots mature, their color shifts to pale yellow green (RHS 194D) and prominent lenticels become visible as raised, light yellow-colored (RHS 162C) corky circles. ‘K207’ as shown in FIG. 9 displays large oval to circular lenticels averaging 2.5 mm in diameter; a characteristic that contrasts with other similar Pongamia cultivars.
Leaves:
   General.—Alternate, imparipinnate with long slender leafstalk, and hairless. About ⅔ (two-thirds) of leaves show 5 leaflets, and about ⅓ (one-third) of leaves shows 7 leaflets.
   Size and shape.—The outline of a compound leaf is roughly oval to elliptic, with an average length of 30 cm and an average width of 16 cm.
   Leaflets.—Blades of individual leaflets are ovate to cordate in shape with a cuspidate to mucronate apex. Leaflet venation tends to be arcuate. Blades of terminal leaflets have an average length of 12 cm and an average width of 8 cm. The smallest leaflet on a leaf tends to be a basal leaflet with an average length of 10 cm and an average width of 7 cm.
   Leaflet color, surface, and texture.—Leaflets are glabrous on both abaxial and adaxial surfaces. Young leaves exhibit a strikingly glossy cuticle on their adaxial surface. The adaxial (upper) and abaxial (lower) surfaces of a young leaf are strong yellow green in color (RHS 144A for adaxial surface, and RHS 141A for abaxial surface). The adaxial surface of an old leaf is moderate dark yellowish green in color (RHS 139A), and the abaxial surface of an old leaf is moderate olive green in color (RHS 146A) with prominent veins beneath. Leaflets turn a darker shade of green as they age and become increasingly sclerophyllous.
   Stipules.—A pair of small stipules subtends the youngest leaves on a shoot. They remain for only a short duration before they dehisce.
Flowers and inflorescences:
   General.—Hermaphroditic florets are borne on an indeterminate inflorescence.
   Inflorescence structure.—Several florets are clustered into a fascicle node, which are generally evenly distributed along a rachis. The entire inflorescence is a pseudoraceme. Most inflorescences are unbranched, but some are branched.
   Position in crown.—‘K207’ has compact inflorescences with an average length of 11 cm. There are an average of 3 inflorescences per lateral shoot. Inflorescences are borne in the basal leaf axils of the current year's vegetative flush, near the distal end of the prior year's shoot growth. Each rachis may hold an average 80 florets.
   Florets.—Emerge acropetally from a fused ovoid calyx with entire margins and glabrous texture. The calyx has an average depth of 4.0 mm and width of 4.5 mm, and is moderate brown in color (RHS 16A). The average length of pedicles is 7.0 mm and the average diameter is 0.5 mm. Pedicels can be dark purple (RHS 83A). Zygomorphic florets consist of 2 keel, 2 wing, and a standard (or banner) petal, and have a depth of 12.1 mm and a diameter of 10.5 mm. The banner petal has a tint of light purplish pink color (RHS 62C) towards the edges, and a strong yellow (RHS N144B) streak in the middle. The banner petal's apex does not have a split notch, and the base is rounded. Wing petals are typically 7.7 mm long and the exposed part is very light purple in color (RHS 75C). The two keel petals are fused together enclosing the stamens and pistil, it is white in color (RHS NN155D), and the distal edges display dark greyish yellow color (RHS 199D). The 10 stamens are fused and enclose the pistil.
   Flowering period.—Relative to other Pongamia cultivars in the general area, ‘K207’ tends to flower about mid-range into the flowering period (from November to December in southeastern Queensland, Australia; and early June in Florida, USA).
Fruits:
   General.—Only one of the two ovules develop into a seed in most of the pods. Immediately after fertilization, the peduncular ovule usually aborts and remains as a small unfilled seed. Only the stigmatic ovule develops into seed, thus pods usually bear a single seed.
   Shape and color.—Developing fruits first appear as moderate yellow green (RHS 146C) flattened pods, expanding in size into a moderate orange yellow (RHS 164B) woody appearance pods when mature.
   Crown distribution.—Pods can occur throughout the crown, but are most prevalent towards the outer portions of the crown.
   Fruit clusters.—Pods within a cluster are connected by the rachis of the inflorescence from which they developed, with an average of 5 pods/bunch.
   Pod dimensions.—Average length of the pod is 33.4 mm, average width of the pod is 18.1 mm, and average thickness of the pod is 8.3 mm. Pods range in shape from oval to half-moon, with an acuminate tip, and average 3.9 g in weight.
Seeds:
   General.—Typically borne singly in pods. Sometimes an aborted (and much smaller) seed occurs alongside a fully-formed seed. About 1-5% of the pods may have two seeds.
   Seed coat.—Seeds are covered by a thin seed coat that is usually brownish orange in color (RHS 164A) when matured.
   Seed size.—Average seed length is 23.0 mm, average seed width is 15.1 mm, and average seed thickness is 6.4 mm. Average seed weight is 2.1 g.
   Seed oil.—Oil content is 40.6% on a dry-weight basis, which is higher than other similar Pongamia cultivars.
   Nut crop.—Mostly a regular bearer (generally in 2 of 3 years).
   Crop frequency.—Most Pongamia cultivars rarely produce abundant nut crops across multiple years. ‘K207’ bears pods more regularly than many other cultivars, generally producing a heavier crop in 2 out of 3 years, and a somewhat lighter crop in the third year.
   Ripening.—Fruit ripening begins in late Oct. and extends into Dec. in southeast Queensland, Australia, and from Jun.-Jul. in Florida, USA. If undisturbed, pods remain in the crown for several weeks after they ripen.

### COMPARISON WITH SIMILAR VARIETIES

Unlike Pongamia cultivars ‘K128b (U.S. Plant Pat. No. 26,062), ‘K140’ (U.S. Plant Pat. No. 26,060), and ‘K606’ (U.S. Plant Pat. No. 26,061), ‘K207’ has a tendency to produce higher yields of pods in about 2 of 3 years, with pod yields in the third year being somewhat less. This pattern of pod yield tends to resemble the yield pattern of cultivar ‘K206’ (U.S. Plant patent application pending), although not necessarily during coincident years.

Seed oil content of ‘K207’, at 40.6% (dry-weight basis), is higher than ‘K206’ (38.4%), ‘K128b’ (38.5%), and ‘K606’0 (38.6%), and is less than that of ‘K140’ (41.4%).

Inflorescences of ‘K207’ are compact (short rachis) and generally contain substantial numbers of florets (averaging 80), which is lower than in ‘K206’, and more than in cultivars ‘K128b’, ‘K140’, and ‘K606’.

‘K207’ has semi-pendulous branchlets (similar to 1 K128b) compared to strongly pendulous ‘K140’, which contrasts with the upright branchlets in ‘K606’ and ‘K206’. This semi-pendulous tendency of branchlets gives trees a more closed canopy, which thereby allows less sunlight to reach leaves deeper in the canopy.

‘K207’ shows unusually large lenticels on the surface of the previous season's branch and other older branches. The branching pattern of the current season's wood is also unusual in ‘K207’, which shows almost every axillary bud developing into a current season's branchlet. The large lenticels and the regular branching pattern are in sharp contrast to ‘K206’, ‘K128b’, ‘K140’, and ‘K606’.

Leaves on ‘K207’ trees include a mix of about ⅔ (two-thirds, or approximately 66%) with 5 leaflets and the remaining ⅓ (one-third, or approximately 33%) with 7 leaflets.

This contrasts with cultivars ‘K128b’, ‘K140’, and ‘K206’ in which leaves with 7 leaflets are relatively common.

We claim:

1. A new and distinct variety of Pongamia pinnata (L) Pierre tree named ‘K207’ as shown and described herein.
